(19) United States
(12) Patent Application Publication
(10) Pub. No.: US 2015/0219641 A1
(43) Pub. Date: Aug. 6, 2015

(54) ANALYSIS CARRIER AND MANUFACTURING METHOD AND USE METHOD THEREFOR

(71) Applicant: SUMITOMO BAKELITE CO., LTD., Shinagawa-ku (JP)

(72) Inventor: Takayuki MATSUMOTO, Shinagawa-ku (JP)

(73) Assignee: SUMITOMO BAKELITE CO., LTD., Shinagawa-ku (JP)

(21) Appl. No.: 14/687,637

(22) Filed: Apr. 15, 2015

Related U.S. Application Data
(63) Continuation of application No. PCT/JP2013/074272, filed on Sep. 9, 2013.

(30) Foreign Application Priority Data
Oct. 19, 2012 (JP) 2012-231599

(51) Int. Cl. G01N 33/543; G01N 33/552; C08F 230/08; G01N 33/545

(52) U.S. Cl. CPC G01N 33/54353; G01N 33/545; G01N 33/552; C08F 230/08

(57) ABSTRACT

An analysis carrier for trapping a biologically active substance includes a carrier body having a carrier surface on which one or more polymers are immobilized. The polymer includes a first repeating unit and a second repeating unit, the first repeating unit has a side chain including a functional group of a betaine structure, and the second repeating unit has a side chain whose terminal group is an active ester group.

### CROSS-REFERENCE TO RELATED APPLICATIONS

The present application is a continuation of and claims the benefit of priority to International Application No. PCT/JP2013/074272, filed Sep. 9, 2013, which is based on and claims the benefit of priority to Japanese Patent Application No. 2012-231599, filed Oct. 19, 2012. The entire contents of these applications are incorporated herein by reference.

### BACKGROUND OF THE INVENTION

1. Field of the Invention

The present invention relates to an analysis carrier for immobilizing a biologically active substance, a manufacturing method of the carrier, and a use method for the analysis carrier having a biologically active substance immobilized on the carrier.

2. Description of Background Art

An analysis carrier for immobilization of a biologically active substance may be provided in a granular, substrate, fiber, filter, film, or sheet form. In a case where the carrier is granular, the carrier is filled in a column or vessel, and may be used to react, separate, or purify the biologically active substance, for example. Alternatively, the carrier may also be used as a diagnostic drug. In addition, in a case where the carrier is a substrate, it may be used as a diagnostic tool similar to the above, for example. Moreover, in a case where the carrier is a fiber, filter, film, or sheet, the carrier can be used as a large quantity separation or purification tool, for example.

### SUMMARY OF THE INVENTION

According to one aspect of the present invention, an analysis carrier for trapping a biologically active substance includes a carrier body having a carrier surface on which one or more polymers are immobilized. The polymer includes a first repeating unit and a second repeating unit, the first repeating unit has a side chain including a functional group of a betaine structure, and the second repeating unit has a side chain whose terminal group is an active ester group.

According to another aspect of the present invention, a method of manufacturing an analysis carrier includes mixing in a solvent a carrier substrate material and a polymerizable monomer such that one or more polymers are formed from the polymerizable monomer and immobilized on a surface of the carrier substrate material, and drying the carrier substrate material having the polymer. The carrier substrate material has a polymerizable functional group or a chain transfer group introduced to the surface before the mixing with the polymerizable monomer, the polymer includes a first repeating unit and a second repeating unit, the first repeating unit has a side chain including a functional group of a betaine structure, and the second repeating unit has a side chain whose terminal group is an active ester group.

### DETAILED DESCRIPTION OF THE EMBODIMENTS

Hereafter, an analysis carrier, and a manufacturing method and use method therefor according to embodiments of the present invention are described.

An analysis carrier according to one embodiment of the present invention is a carrier having a function immobilizing a biologically active substance. Specifically, a polymer is immobilized on a carrier surface, the polymer including a first repeating unit having a functional group having a betaine structure on a side chain; and a second repeating unit having an active ester group on an end of a side chain.

The first repeating unit having the functional group having the betaine structure on the side chain plays a role of inhibiting non-specific adsorption of proteins or the like other than a detection target.

In addition, the second repeating unit having the active ester group on the end of the side chain is capable of bonding various biologically active substances having an amino group to a carrier easily and with a high level of efficiency. This enables the detection target specifically bonded to the biologically active substance to be trapped.

In light of this, the analysis carrier has a function trapping various biologically active substances, and the carrier having the immobilized biologically active substance can detect the detection target with a high degree of selectivity.

Herein, examples of the biologically active substance can include a protein such as an enzyme, an antibody, lectin, a receptor, protein A, protein G, protein A/G, avidin, streptavidin, NeutrAvidin, glutathione-S-transferase, or glycoprotein; a peptide; an amino acid; a hormone; a nucleic acid; a sugar chain such as a sugar, oligosaccharide, polysaccharide, sialic acid derivative, or sialylated carbohydrate chain; a lipid; a low molecular weight compound; an organic polymer substance other than those named above; an inorganic substance; or a cointegrate of these, or a molecule forming a virus or a cell.

A first repeating unit having a functional group with a betaine structure on the side chain is derived from a carboxy betaine monomer, a sulfobetaine monomer, a phosphobetaine monomer, or the like for example, and a monomer having a phosphoryl choline structure is also one kind of monomer having a betaine structure. The betaine structure enables non-specific adsorption of protein contained in blood serum or cell lysate, for example, to be largely inhibited. Of these, a monomer having a phosphoryl choline structure is highly preferred for its non-specific adsorption inhibition effect. In addition, the first repeating unit preferably includes a polymerizable group in addition to the functional group having the above-noted betaine structure. The polymerizable group is preferably an ethylene-based unsaturated polymerizable group. In other words, the first repeating unit is preferably an ethylene-based unsaturated polymerizable monomer having a functional group with a betaine structure.

As shown in Formula [1] below, an ethylene-based unsaturated polymerizable monomer having a functional group with a betaine structure is preferably a compound in which a functional group having a (meth)acrylic group and a betaine structure is bonded directly or via a hydrocarbon chain of between 1 and 20 carbons, which may be interrupted by —O—, —S—, —NH—, —CO—, or —CONH—. Moreover, in Formula [1] below, a hydrocarbon chain where X is 0 carbons refers to an oxygen atom in the formula (—O—) and R₂ bonding directly, and X being a single bond.

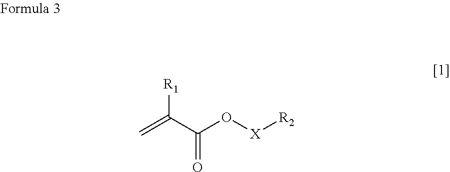

(In the formula, R₁ represents a hydrogen atom or methyl group, and R₂ represents a functional group having a betaine structure. X represents a hydrocarbon chain of between 0 and 20 carbons, which may be interrupted by —O—, —S—, —NH—, —CO—, or —CONH—.)

Concrete examples of a carboxy betaine monomer may include dimethyl(2-methacryloyloxyethyl)(2-carboxylate ethyl)aminium, dimethyl(2-acryloyloxyethyl)(2-carboxylate ethyl)aminium, dimethyl(2-methacryloyloxyethyl)(3-carboxylate propyl)aminium, dimethyl(2-acryloyloxyethyl)(3-carboxylate propyl)aminium, dimethyl(2-methacryloyloxyethyl)(4-carboxylate butyl)aminium, dimethyl(2-acryloyloxyethyl)(4-carboxylate butyl)aminium, dimethyl(2-methacryloyloxyethyl)(carboxylate methyl)aminium, dimethyl(2-acryloyloxyethyl)(carboxylate methyl)aminium, or the like.

Concrete examples of a sulfobetaine monomer may include dimethyl(2-methacryloyloxyethyl)(2-sulfonate ethyl)aminium, dimethyl(2-acryloyloxyethyl)(2-sulfonate ethyl)aminium, dimethyl(2-methacryloyloxyethyl)(3-sulfonate propyl)aminium, dimethyl(2-acryloyloxyethyl)(3-sulfonate propyl)aminium, dimethyl(2-methacryloyloxyethyl)(4-sulfonate butyl)aminium, dimethyl(2-acryloyloxyethyl)(4-sulfonate butyl)aminium, dimethyl(2-methacryloyloxyethyl)(sulfonate methyl)aminium, dimethyl(2-acryloyloxyethyl)(sulfonate methyl)aminium, or the like.

Concrete examples of a phosphobetaine monomer may include dimethyl(2-methacryloyloxyethyl)(2-phosphonate ethyl)aminium, dimethyl(2-acryloyloxyethyl)(2-phosphonate ethyl)aminium, dimethyl(2-methacryloyloxyethyl)(3-phosphonate propyl)aminium, dimethyl(2-acryloyloxyethyl)(3-phosphonate propyl)aminium, dimethyl(2-methacryloyloxyethyl)(4-phosphonate butyl)aminium, dimethyl(2-acryloyloxyethyl)(4-phosphonate butyl)aminium, dimethyl(2-methacryloyloxyethyl)(phosphonate methyl)aminium, dimethyl(2-acryloyloxyethyl)(phosphonate methyl)aminium, or the like.

In addition, concrete examples of a polymerizable unsaturated monomer having a phosphorylcholine structure may include 2-(meth)acryloyloxyethyl phosphorylcholine, 2-(meth)acryloyloxyethoxyethyl phosphorylcholine, 6-(meth)acryloyloxyhexyl phosphorylcholine, 10-(meth)acryloyloxyethoxynonyl phosphorylcholine, 2-(meth)acryloyloxypropyl phosphorylcholine, 2-(meth)acryloyloxybutyl phosphorylcholine, or the like. Of these, 2-(meth)acryloyloxyethyl phosphorylcholine is most preferred due to being readily available.

Next, the second repeating unit having an active ester group on the end of the side chain is preferably derived from an ethylene-based unsaturated polymerizable monomer having an active ester group on an end, for example.

As shown in Formula [2] below, an ethylene-based unsaturated polymerizable monomer having an active ester group on an end is preferably a compound in which a (meth)acrylic group and an active ester group are bonded via a chain of an alkylene group or a 1 to 10 carbon alkylene glycol residue Y.

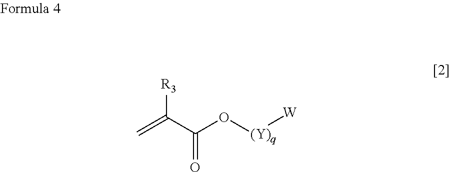

(In the formula, R₃ represents a hydrogen atom or methyl group, and Y represents a 1 to 10 carbon alkylene glycol residue or an alkylene group. W represents an active ester group. q is an integer between 1 and 100. In a case where q is an integer between 2 and 100, the repeating Y may be the same or different.)

In a case where Y in Formula [2] above is an alkylene group, q is an integer between 1 and 100, preferably between 1 and 20, more preferably between 1 and 10, and most preferably between 1 and 6. When the value of q is too great, non-specific adsorption of protein increases.

When Y in Formula [2] is an alkylene glycol residue (polyoxyalkylene group), there are between 1 and 10 carbons in the alkylene glycol residue Y, preferably between 1 and 6, more preferably between 2 and 4, still more preferably between 2 and 3, and most preferably 2. When the number of carbons is within this range, there is particularly superior inhibition of non-specific adsorption. In addition, the number of repetitions q of the alkylene glycol residue Y is not particularly limited, but is preferably an integer between 1 and 100, more preferably an integer between 2 and 100, still more preferably an integer between 2 and 95, and most preferably an integer between 4 and 90. When the number of repetitions q is within this range, there is particularly superior inhibition of non-specific adsorption.

The “active ester group” used in the present invention refers to an ester group that includes an electron withdrawing group with high acidity in one substitution group of an ester group and that activates in response to a nucleophilic reaction (in other words, a highly reactive ester group). This meaning is in common use in various chemosynthesis fields such as polymer chemistry, peptide synthesis, and the like. In practice, phenol esters, thiophenol esters, N-hydroxyamine esters, heterocyclic hydroxy compound esters, and the like are examples of active ester groups having far greater activity as compared to alkyl esters and the like.

Examples of such an active ester group may include a p-nitrophenyl active ester group, N-hydroxysuccinimide active ester group, succinimide active ester group, phthalic imide active ester group, 5-norbornene-2,3-dicarboxyimide active ester group, and the like, where the p-nitrophenyl active ester group or N-hydroxysuccinimide active ester group are preferred, and the p-nitrophenyl active ester group is most preferred.

A copolymerization ratio of the first repeating unit and the second repeating unit of the polymer described above (first repeating unit/second repeating unit) is not particularly limited; however, the copolymerization ratio is preferably between 97/3 and 5/95, and between 90/10 and 10/90 is particularly preferred. When the copolymerization ratio is within this range, inhibition of non-specific adsorption is particularly effective, and there is a superior biologically active substance immobilization effect and superior trapping effect on a biological substance specifically bonding with the biologically active substance.

The copolymerization ratio can be calculated by, for example, evaluating an elemental composition using X-ray photoelectron spectroscopic analysis (XPS).

The polymer is preferably a random copolymer that includes a first repeating unit and a second repeating unit. Accordingly, the polymer can be act effectively to disperse an active ester group present on the end of a side chain of the second repeating unit. The weight-average molecular weight of the polymer is not particularly limited, but is preferably between 5000 and 1,000,000, and between 10,000 and 100,000 is particularly preferred. When the weight-average molecular weight is within this range, handling during synthesis is favorable, and non-specific adsorption can be effectively inhibited.

In the analysis carrier, a polymer formed from a first repeating unit, a second repeating unit, and a unit having a silane coupling agent on a side chain may be bonded to the substrate (carrier) via the silane coupling agent. Thereby, the polymer can be prevented from separating from the carrier.

Examples of the unit having a silane coupling agent on a side chain may include a unit derived from methacryloxypropyl dimethylmethoxysilane, methacryloxypropyl dimethylethoxysilane, methacryloxypropyl methydimethoxysilane, methacryloxypropyl methyl diethoxysilane, methacryloxypropyl trimethoxysilane, methacryloxypropyl triethoxysilane, 3-mercaptopropyl trimethoxysilane, 3-mercaptopropyl triethoxysilane, 3-mercaptopropyl methyldimethoxysilane, 3-mercaptopropyl methyldiethoxysilane, 3-mercaptopropyl dimethylmethoxysilane, 3-mercaptopropyl dimethyl ethoxysilane, or mercaptoethyl triethoxysilane.

In a case where the carrier is an inorganic oxide, by using the silane coupling agent, the carrier and the polymer can be easily coupled by a coupling reaction between the hydroxyl group on the carrier surface and the polymer.

By using the silane coupling agent, the polymer can be prevented from separating from the carrier surface, and therefore during use as an analysis carrier, the polymer can be prevented from dissolving during repeated heating processes and cleaning steps. Moreover, a chemically and physically stable analysis carrier can be provided in which a reduction in non-specific adsorption components and active ester groups accompanying separation of the polymer is inhibited.

In addition, by preventing reduction of the active ester group, an amount of immobilization of the biologically active substance can be maintained at a high level, and therefore a trapped amount of a substance (detection target) selectively trapped by the biologically active substance increases. Moreover, because the reduction in non-specific adsorption components is inhibited, non-specific adsorption of proteins other than the detection target is reduced, and an analysis carrier having a high S/N ratio can be provided.

A substrate material of the carrier is not particularly limited, and both organic and inorganic materials can be used. Examples of an organic material include, in addition to porous agarose granules used as an affinity chromatography carrier (trade name: Sepharose) and dextran granules (trade name: Sephadex), polyacrylamide gel (trade name: Bio-Gel P, Biorad Co.), polystyrene, ethylene-maleic anhydride copolymer, polymethyl methacrylate, polyolefin, polystyrene, polyethylene, polycarbonate, polyamide, various resin materials such as acrylic resin, and the like.

Examples of an inorganic material may include gold, silver, platinum, palladium, iridium, rhodium, osmium, iron, copper, cobalt, aluminum, and alloys or inorganic oxides of the same. Of these, an inorganic oxide is preferred due to having a high degree of material strength. Of these, silicon oxide is easily obtained, and is most preferred.

The carrier can have any form, such as granular, substrate, fiber, filter, film, or sheet. Of these, when the carrier is granular, the polymer is readily fixated to the surface, and therefore this is preferred. Examples of a substrate carrier may include a flat plate-like substrate having a microscope slide shape, a multiwell plate, or the like.

When the carrier is granular, an average particle size of the carrier may be selected as appropriate according to objective and application. In particular, when the carrier is inorganic, controlling particle size is easy as compared to a method of manufacturing organic granules with emulsion polymerization or suspension polymerization, in which controlling particle size is difficult.

Specifically, although particle size of the granular carrier differs by application, an average particle size for the granular carrier used in the analysis carrier is preferably several nm to 100 μm. In particular, 100 nm to 50 μm is preferred, and 1 μm to 40 μm is most preferred. When the average particle size of the carrier is within this range, a particularly superior balance is achieved between the amount of biologically active substance trapped and quality of handling. The average particle size can be measured using a particle size analyzer, for example.

### (Manufacture of Polymer Immobilizing Carrier)

Manufacture of the polymer immobilizing carrier is described. A method of manufacturing the carrier is not particularly limited; however, due to ease of synthesis, preferably a silane coupling agent having a polymerizable functional group or chain transfer group is fixated to a carrier surface, and a polymerizable monomer having a functional group with a betaine structure on the side chain and a mixture which includes a polymerizable monomer having an active ester group and the carrier are radical polymerized in a solvent in the presence of a polymerization initiator.

The silane coupling agent described above can be preferably employed as a silane coupling agent having a polymerizable functional group or chain transfer group.

The solvent may be anything dissolving the various monomers, e.g., the ethylene-based unsaturated polymerizable monomer, examples of which may include 2-butanone, methanol, ethanol, t-butyl alcohol, benzene, toluene, tetrahydrofuran, dioxane, dichloromethane, chloroform, and the like. These solvents may be used singly or in combination of two or more.

The polymerization initiator may be any typical radical initiator, examples of which may include an azo compound such as 2,2′-azobisisobutyl nitrile (hereafter, “AIBN”), 1,1′-azobis(cyclohexane-1-carbonitrile), and the like; an organic peroxide such as benzoyl peroxide or laurel peroxide; and the like.

A chemical structure of the polymer substance may have any coupling scheme, such as random, block, or graft.

In addition, by fixating the pre-polymerized polymer substance on the carrier surface, the polymer immobilizing carrier may be manufactured. In such a case, for example, the solution of the polymer substance is prepared, then applied to the carrier surface using a method such as dipping, blowing, or the like, after which the solution is dried at room temperature or heated.

A concentration of the polymer substance solution is not particularly limited, and is preferably 0.05 mass % or more, more preferably 0.1 to 70 mass %, still more preferably 0.1 to 50 mass %, and most preferably 0.3 to 50 mass %. When the concentration of the polymer substance in the polymer substance solution falls below a lower limit value, an amount of polymer substance applied to the carrier surface is reduced. Therefore, an amount of immobilized biologically active substance is reduced, and also a trapping effect on a biological substance specifically bonding with the biologically active substance (i.e., the target substance) is reduced. Moreover, an effect inhibiting non-specific adsorption of proteins or the like to the carrier is also reduced. Therefore, in a case where the concentration of the polymer substance solution falls below the lower limit, there is a chance that the characteristic of selectively trapping the target substance may not be fully achieved.

In addition, when the polymer substance is applied to the carrier, the concentration thereof may be adjusted to a predetermined concentration ahead of time; however, the polymer substance solution can also be applied to the carrier while being concentrated in the application step. When a low concentration polymer substance solution is used for application to a carrier (granules), viscosity of the solution is low, and therefore the solution may readily infiltrate the carrier surface, which has a fine shape (such as fine holes). This is advantageous in that the polymer substance solution is able to access nooks and crannies of the carrier surface; however, the carrier surface may not be adequately coated by the polymer substance due to the low concentration. Meanwhile, when a high concentration polymer substance solution is used, an increase in the amount of polymer substance applied to the carrier surface can be expected; however, by increasing surface tension of the solution, wettability of the carrier is reduced, and manipulability deteriorates. Therefore, in order to adequately coat the polymer substance on a carrier having a complex surface shape, a method of applying the polymer substance solution while increasing concentration from a low concentration solution is preferred. The method of increasing concentration is not particularly limited, and any method such as heat evaporation, vacuum concentration, or the like can be selected as desired.

Examples of a solvent used in the polymer substance solution are not particularly limited so long as the polymer substance is dissolved, and examples can include alcohols such as ethanol, methanol, isopropanol, n-butanol, t-butyl alcohol, n-pentanol, and cyclohexanol; benzene, toluene, tetrahydrofuran, dioxane, dichloromethane, chloroform, acetone, methyl acetate, ethyl acetate, butyl acetate, methyl ethyl ketone, methyl butyl ketone, ethylene glycol monoethyl ether, ethylene glycol monomethyl ether, ethylene glycol monobutyl ether, and cyclohexanone. These solvents may be used singly or in combination of two or more kinds. Of these, ethanol and methanol are highly versatile and easily dried, and are therefore preferred.

In addition, conditions for covalently bonding the polymer substance with a functional group of the carrier surface using the contained silane coupling agent can be selected as desired according to the silane coupling agent. For example, in the case of a polymer substance having alkoxysilane, a silanol group produced by hydrolysis forms a covalent bond by dehydration condensation with a hydroxyl group, amino group, carbonyl group, silanol group, or the like of the carrier surface. The covalent bond formed by dehydration condensation of the silanol group has a characteristic of being difficult to hydrolyze. Therefore, the polymer substance immobilized on the surface of the grain serving as the nucleus is not readily dissolved, and is not stripped from the grain serving as the nucleus. Dehydration condensation of the silanol group is promoted by a heating process. The heating process is preferably performed within a temperature range where the polymer substance is not transformed by heat, e.g., between 60 and 180° C. for between 5 minutes and 24 hours.

In a case where an organic solvent having high polarity is used, such as ethanol or methanol, or in a case where the polymer substance itself is highly hydrophilic, hydrolysis of an alkoxysilyl group occurs due to hydrogen contained in the solvent or hydrogen in the atmosphere after application; therefore, even without performing a special hydrolysis step, the polymer substance can often be immobilized simply by heating the carrier. In a case where hydrolysis is insufficient, a mixed solution containing water in an organic solvent may also be used. Supplying the water in a theoretical amount for producing the silanol group by hydrolysis is sufficient; however, when ease of preparing the solution is considered, water content is preferably kept at 15 mass % or less. When the water content becomes greater, the polymer solution may be insoluble in a solvent.

When the polymer substance is immobilized on the carrier surface, so long as there is a functional group capable of reacting with the polymer substance on the carrier surface, it can be used in that state; however, when such a functional group is absent or scarce, the carrier surface is preferably activated. A method of activation is not particularly limited, and examples may include a method using alkoxysilane as a surface processor; a method of processing using an acid or alkali; a method of plasma processing in conditions such as an oxide atmosphere, an argon atmosphere, a nitrogen atmosphere, an air atmosphere, or the like; or a method of processing using an excimer laser of ArF, KrF, or the like. In a case where the carrier is granular, a method using alkoxysilane and/or a method of processing using an acid/alkali is preferred.

Examples of the alkoxysilane used as the surface processing agent are not particularly limited and may include dialkoxysilane, trialkoxysilane, tetralkoxysilane, and the like. Of these, the tetralkoxysilane, which has the greatest number of alkoxysilyl groups per molecule, is preferably used. Specific examples of the tetralkoxysilane may include tetramethoxysilane, tetraethoxysilane, tetrapropoxysilane, tetrabutoxysilane, tetraphenoxysilane, and the like. A tetralkoxysilane with a comparatively small molecular weight can impart a greater number of alkoxysilyl groups to the surfaces of the grain serving as the nucleus; therefore, tetramethoxysilane, tetraethoxysilane, and tetrapropoxysilane having an alkoxysilyl group with three or fewer carbons are preferred, and tetraethoxysilane is more preferred due to availability. These tetroxysilanes may be used singly or in combination of two or more kinds.

A method of activating the carrier surface using alkoxysilane is described. Conditions and the like are not particularly limited, and a carrier is, for example, immersed in an alkali catalyst and a solution containing an alcohol so as to be between 0.05 and 10 mass %, and is performed while adding alkoxysilane dissolved in alcohol to the dispersion medium. A usage ratio of the carrier and alkoxysilane is not particularly limited; however, in a case where the carrier is granular, a ratio of between 0.01 and 10 mmol alkoxysilane per 1 g may be used. Examples of the alcohol included in the dispersion medium and the alcohol dissolving alkoxysilane are not particularly limited, and ethanol, methanol, isopropanol, t-butyl alcohol, and the like may be used singly or in combination of two or more kinds. Of these, methanol, which is readily dried and inexpensive, is preferred.

After an alkoxysilane solution is added, typically it is stirred at between 0 and 50° C. for between 5 and 30 minutes to perform surface processing. The obtained carrier is cleaned, after which it is dried.

In the above-noted processing conditions, dehydration condensation occurs between the alkoxysilyl group of the alkoxysilane and the functional group of the carrier surface. At this point, dehydration condensation of the alkoxysilyl group to be used in immobilizing the polymer substance is likely to occur simultaneously; therefore, in order for an effect of surface processing using the alkoxysilane to be fully achieved, after the above-noted processing, it is effective to perform processing on the carrier using acid/alkali. In particular, processing using acid is preferred for hydrolysis of a siloxane bond produced by dehydration condensation of the alkoxysilyl group. A method for processing the carrier surface using acid is not particularly limited, and can, for example, be performed by immersing the carrier obtained by the above-noted process in an acid of between 0.01 and 3 N for between 1 and 5 hours. Various common inorganic acids and/or organic acids can be used as the acid used in the processing. Examples of the inorganic acid may include sulfuric acid, nitric acid, hydrochloric acid, hydrofluoric acid, and the like, while examples of the organic acid may include formic acid, acetate, benzoic acid, and the like. An inorganic acid is preferred that is capable of imparting comparatively strict processing conditions for completing processing in a short amount of time, and of these hydrochloric acid is more preferred due to being readily removed after processing due to high volatility, and being comparatively easy to handle.

### (Immobilization of Biologically Active Substance)

When immobilizing a biologically active substance on a carrier, a method is preferred that adheres a fluid in which the biologically active substance is dissolved or dispersed. The pH of the fluid in which the biologically active substance is dissolved or dispersed is preferably between 5.0 and 11.0, and is more preferably between 6.0 and 10. When outside this range, the biologically active substance may be denatured/dissolved.

After adhesion of the biologically active substance, the carrier is preferably processed with a low molecular weight substance having an amino group such as amino ethanol, and the unreacted active ester group is deactivated. Due to the properties of the hydrophilic group on the carrier surface, by cleaning with water containing a surfactant or with a buffer solution, non-specific adsorption of substances other than the target substance to the solid-phase surface can be inhibited.

The biologically active substance is at least one selected from among a protein such as an enzyme, an antibody, a lectin, a receptor, protein A, protein G, protein A/G, avidin, streptavidin, NeutrAvidin, glutathione-S-transferase, or a glycoprotein; a peptide; an amino acid; a hormone; a nucleic acid; a sugar chain such as a sugar, oligosaccharide, polysaccharide, sialic acid derivative, or sialylated carbohydrate chain; or a lipid, a low molecular weight compound, an organic polymer substance other than those named above, an inorganic substance, or a cointegrate of these, or a molecule forming a virus or a cell.

### EXAMPLES

A detailed description of the present invention follows based on examples and a comparative example; however, the present invention is not limited to these.

### Example 1

### Case Where a Silane Coupling Agent Having a Polymerizable Functional Group is Immobilized on a Carrier Surface, the Beads are Dispersed in a Monomer Solution, and a Polymer Substance is Synthesized

### Synthesis of p-nitrophenyloxycarbonyl-polyethylene glycol methacrylate (MEONP)

After dissolving 0.01 mol of polyethylene glycol monomethacrylate (Blenmer PE-200 (n=4), mfd. by Nippon Oil & Fats Co., Ltd.) in 20 mL of chloroform, the solution was cooled to −30° C. While holding at −30° C., 0.01 mol of pre-manufactured p-nitrophenyl chloroformate (mfd. by Aldrich) and 0.01 mol of triethylamine (mfd. by Wako Pure Chemicals Co., Ltd.), as well as 20 mL chloroform uniform solution, were slowly dripped into the solution. After reacting for one hour at −30° C., the solution was then stirred for two hours. After this, salt was eliminated from the reaction solution using filtration, then the solvent was distilled to obtain p-nitrophenyloxycarbonyl-polyethylene glycol methacrylate (MEONP). The obtained monomer was measured using 1H-NMR in heavy chloroform solvent, and was confirmed to contain an average of 4.5 units of ethylene glycol residue.

### (Introduction of Silane Coupling Agent to Carrier Surface)

To a mixed solution of 50 mL pH 3.0 acetic acid solution and 50 mL ethanol was added 13 g of methacryloxypropyl dimethyl methoxysilane (mfd. by Gelest Co., SIM 6486.5), and the silane coupling agent was hydrolyzed, after which 10 g of silica beads (average particle size 5 μm, pore size 70 Å, mfd. by Fuji Silysia Co., SMB 70-5) was added and stirred at 70° C. for two hours, after which the silica beads are collected from the reaction solution using suction filtration and heated at 100° C. for one hour. Thereafter, the silica beads were dispersed in ethanol and shaken, after which a supernatant was removed by centrifugation and dried.

### (Immobilization of Polymer Substance on Carrier Surface)

2-methacryloyloxyethylphosphorylcholine (hereafter referred to as MPC monomer, mfd. by Nippon Oil & Fats Co., Ltd.) and the previously synthesized MEONP were dissolved in a mixed solvent of ethanol and methyl ethyl ketone, and a monomer mixed solution was produced. Total monomer concentration was 0.8 mol/L, the molar ratios of each being, in the order of MPC monomer and MEONP, 80:20, 50:50, and 20:80. AIBN was then added so as to be 0.08 mol/L, and was stirred until uniform. Thereafter, 10 g of the silica beads processed with methacryloxypropyl dimethyl methoxysilane were added and were reacted for 22 hours at 70° C. in an argon gas atmosphere. Next, the silica beads were collected from the reaction solution using centrifugation, were dispersed in dimethylsulfoxide, and were well shaken, after which the beads were collected using suction filtration and dried.

### Example 2

### Case Where Polymer Substance is Pre-Synthesized and Applied to Carrier

### (Synthesis of Polymer Substance)

The MPC monomer, MEONP, and 3-methacryloxypropyl dimethyl methoxysilane (MPDMS) were dissolved in a mixed solvent of ethanol and methyl ethyl ketone, and a monomer mixed solution was produced. Total monomer concentration was 0.8 mol/L, the molar ratios of each being, in the order of MPC monomer, MEONP, and MPDMS, 47:47:6. AIBN was then added so as to yield 0.08 mol/L, and was stirred until uniform. Thereafter, it was reacted for four hours at 60° C. in an argon gas environment, after which the reaction solution was dripped into a mixed solvent of diethyl ether and chloroform, precipitate was collected and was again dissolved in a mixed solvent of ethanol and methyl ethyl ketone, and was prepared at a concentration of 0.3 wt %.

### (Coating of Silica Beads)

Silica beads having an average particle size of 5 microns were immersed in the polymer substance solution, and were well mixed by a vortex mixer. The mixed fluid was concentrated using a rotary evaporator. Moreover, the beads were collected using suction filtration and were well dried, after which heat processing was performed at 100° C. for two hours. Thereafter, the beads were immersed in a mixed solvent of ethanol and methyl ethyl ketone, were well mixed by the vortex mixer, and were cleaned. The beads were collected using suction filtration and dried.

### (Immobilization of Primary Antibody)

One mL of a dipotassium hydrogenphosphate solution of a CRP antibody (mfd. by Abnova) prepared at 50 μg/mL was added to 20 mg each of the grains obtained in Examples 1 and 2, and were inversion mixed for one night at room temperature. The solutions were cleaned three times with PBS containing 0.05% Tween20. Moreover, the solutions were processed with 0.1 mol/L 2-aminoethanol (solvent: pH 9.5, 0.05 mol/L Tris-HCl buffer solution) at room temperature for one hour, and deactivation of the active ester group was performed.

(Reaction with CRP)

One mL of a PBS solution of CRP prepared at 3 μg/mL was added to 5 mg of the grains to which the CRP antibody was immobilized, and were inversion mixed for one hour at room temperature. After collecting the grains using centrifugation, the grains were cleaned three times with PBS containing 0.05% Tween20.

(Reaction with Secondary Antibody)

One mL of HRP-tagged CRP antibody (mfd. by Abnova) solution prepared at 1 μg/mL was added to the grains reacted with CRP, and were inversion mixed for one hour at room temperature. After collecting the grains using centrifugation, the grains were cleaned three times with PBS containing 0.05% Tween20.

### (Quantity of CRP Trapping)

The grains reacted with the HRP-tagged CRP antibody were dyed using a peroxidase dye kit manufactured by Sumitomo Bakelite Co., Ltd., and a CRP trapping amount was estimated by measuring 450 nm light absorbance.

### COMPARATIVE EXAMPLE

CRP trapping amount measurement similar to that of the examples was performed on grains for which only deactivation with 2-aminoethanol was performed and immobilization of the CRP antibody was not performed.

TABLE 1

| | | Light absorbance at 450 nm | |
|---|---|---|---|
| | MPC/MEONP | Examples | Comparative Example |
| Example 1 | 80/20 | 0.95 | 0.08 |
| | 50/50 | 1.66 | 0.13 |
| | 20/80 | 2.03 | 0.32 |
| Example 2 | 46/46 | 1.21 | 0.22 |

In both of the examples, light absorbance was more greatly elevated than in the comparative example, and the beads to which the CRP antibody was immobilized were understood to be capable of trapping CRP.

In case of applications, the biologically active substance is to be securely immobilized on the carrier surface, and therefore, previously, the mainstream was to immobilize the biologically active substance on a resin using physical chemical adsorption. On the other hand, at present, there is a method that introduces a functional group to the carrier surface and immobilizes the biologically active substance using chemical bonding.

Thereby, detachment of the biologically active substance can be prevented, and the biologically active substance can be securely immobilized regardless of molecular weight or structure.

Patent Document 1 describes a method in which microbeads of resin encasing magnetic bodies are produced by emulsion polymerization, ethylene glycol diglycidyl ether is reacted with a functional group on the bead surfaces, and a monoclonal antibody is coupled. However, this method requires a step of producing the microbeads using polymerization, which is complex. In addition, controlling the required particle diameter and particle size distribution is difficult.

Meanwhile, Patent Document 2 describes a method in which readily-obtained general-purpose resin microbeads are used as a base material, the surfaces of the microbeads are hydrolyzed, hydrophilic spacer molecules are bonded to refined carboxylic acid on the surfaces, then a biologically active substance is bonded to the functional groups of the spacer molecules, and non-specific adsorption is inhibited by the hydrophilia.

The microbeads obtained using this method require using, as a base material, a resin generating carboxylic acid due to hydrolysis. In addition, in a scenario where the microbeads are used to trap a substance having a high affinity for the biologically active substance immobilized by the spacer molecules, there is a significant possibility that non-specific adsorption cannot be inhibited when contact is made with a specimen containing a large amount of biologically-derived impurities.

Patent Document 1: Japanese Patent Laid-open Publication No. 2005-241547

Patent Document 2: Japanese Patent Laid-open Publication No. 2009-031130

In one embodiment, the present invention provides a carrier capable of immobilizing a biologically active substance, and particularly preferably it readily prepares and provides a carrier which can immobilize a biologically active substance, and in which non-specific adsorption is inhibited.

In another embodiment, the present invention provides a carrier in which a biologically active substance is immobilized on an analysis carrier used in immobilizing the biologically active substance.

The present invention includes the following aspects.

(1) An analysis carrier trapping a biologically active substance, in which a polymer is immobilized on a carrier surface, and the polymer includes a first repeating unit having a functional group with a betaine structure on a side chain; and a second repeating unit having an active ester group on an end of the side chain.

(2) The analysis carrier according to (1), in which the functional group with the betaine structure is a phosphorylcholine group.

(3) The analysis carrier according to (1) or (2), in which a layer containing a polymer substance is formed on the carrier surface by introducing a polymerizable functional group or chain transfer group to a surface of the carrier, mixing the carrier with polymerizable components including a polymerizable monomer having a functional group with a betaine structure on a side chain and a polymerizable monomer having an active ester group, then advancing a polymerization reaction.

(4) The analysis carrier according to (3), in which the polymerizable monomer having the functional group with the betaine structure on the side chain includes a monomer expressed by general formula [1] below.

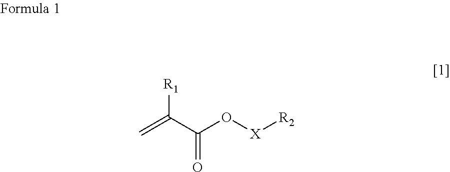

(In the formula, R₁ represents a hydrogen atom or methyl group, and R₂ represents a functional group having a betaine structure. X represents a hydrocarbon chain of between 0 and 20 carbons, which may be interrupted by —O—, —S—, —NH—, —CO—, or —CONH—.)

(5) The analysis carrier according to (3) or (4), in which the polymerizable monomer having the functional group with the betaine structure on the side chain is 2-(meth)acryloyloxyethyl phosphorylcholine.

(6) The analysis carrier according to any of (3) to (5), in which the polymerizable monomer having the active ester group includes a monomer expressed by general formula [2] below.

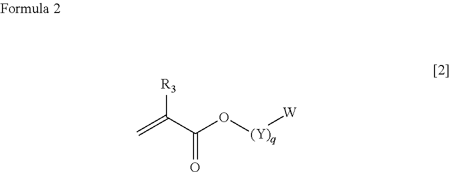

(In the formula, R₃ represents a hydrogen atom or methyl group, and Y represents a 1 to 10 carbon alkylene glycol residue or an alkylene group. W represents an active ester group. q is an integer between 1 and 100. In a case where q is an integer between 2 and 100, the repeating Y may be the same or different.)

(7) The analysis carrier according to (6), in which the active ester group is a group including a p-nitrophenyl group or succinimide group, and an ester bond.

(8) The analysis carrier according to any of (3) to (7), in which the polymerizable functional group introduced to the carrier surface is at least one kind selected from a methacryl group, an acryl group, and a vinyl group.

(9) The analysis carrier according to any of (3) to (7), in which the chain transfer group introduced to the carrier surface is a mercapto group.

(10) The analysis carrier according to any of (1) to (9), in which the carrier is formed by an inorganic material.

(11) The analysis carrier according to (10), in which the inorganic material is formed by an inorganic oxide.

(12) The analysis carrier according to (11), in which the inorganic oxide is silicon oxide.

(13) The analysis carrier according to any of (1) to (12), in which the carrier has a granular, substrate, fiber, filter, film, or sheet form.

(14) The analysis carrier according to any of (3) to (13), in which introduction of the polymerizable functional group or the chain transfer group to the carrier surface is performed in a form of a covalent bond between a silane coupling agent having a polymerizable functional group or a chain transfer group and a functional group of the carrier surface serving as a nucleus.

(15) The analysis carrier according to (14), in which the silane coupling agent having the polymerizable functional group or the chain transfer group is an alkoxysilane having a polymerizable functional group or a chain transfer group.

(16) The analysis carrier according to any of (1) to (15), in which a biologically active substance is immobilized via the active ester group of the layer that includes a polymer substance.

(17) The analysis carrier according to (16), in which the biologically active substance is at least one selected from among at least one protein selected from a group of an enzyme, an antibody, a lectin, a receptor, protein A, protein G, protein A/G, avidin, streptavidin, NeutrAvidin, glutathione-S-transferase, or a glycoprotein; a peptide; an amino acid; a hormone; a nucleic acid; at least one sugar chain selected from a group of a sugar, oligosaccharide, polysaccharide, sialic acid derivative, or sialylated carbohydrate chain; a lipid; a low molecular weight compound; an organic polymer substance other than those named above; an inorganic substance; or a cointegrate of these, or a molecule forming a virus or a cell.

(18) A manufacturing method of the analysis carrier according to any of (1) to (17), the manufacturing method of the analysis carrier including hydrolyzing an alkoxysilane having a polymerizable functional group or a chain transfer group in an acid aqueous solution; next stirring and heating a carrier in an acid aqueous solution containing the alkoxysilane having the polymerizable functional group or chain transfer group; and further heating the carrier after drying.

(19) The manufacturing method of the analysis carrier according to any of (1) to (17), the manufacturing method of the analysis carrier including advancing a polymerization reaction by mixing together in a solvent a polymerizable monomer and the carrier to which the polymerizable functional group or the chain transfer group have been introduced; and drying.

(20) The manufacturing method of the analysis carrier according to (19), in which the polymerization reaction is a radical polymerization reaction.

(21) The manufacturing method of the analysis carrier according to any of (3) to (17), the manufacturing method of the analysis carrier including bringing a solution in which a biologically active substance is dissolved in a phosphate buffer solution into contact with a carrier having a layer containing a polymer substance formed thereon.

(22) The manufacturing method of the analysis carrier according to (21), in which a phosphate concentration of the phosphate buffer solution is between 0.1 M and 5 M.

(23) The manufacturing method of the analysis carrier according to (21) or (22), in which the phosphate includes any of potassium dihydrogenphosphate, sodium dihydrogenphosphate, dipotassium hydrogenphosphate, or disodium hydrogenphosphate.

(24) A use method of the analysis carrier according to any of (21) to (23), in which a target biological substance is collected by bringing the analysis carrier into contact with at least one solution selected from a solution of a target biological molecule, blood, blood plasma, blood serum, disrupted cell suspension, cell culture liquid, and tissue fractionation liquid.

According to an embodiment of the present invention, an analysis carrier capable of immobilizing a biologically active substance can be provided, and in particular enables a carrier to be readily prepared and provided which can immobilize a biologically active substance without requiring a catalyst, and in which non-specific adsorption is inhibited.

### INDUSTRIAL APPLICABILITY

The present invention provides an embodiment of a carrier which is capable of immobilizing a biologically active substance, and specifically is capable of readily preparing and providing a carrier which can immobilize a biologically active substance without requiring a catalyst, and in which non-specific adsorption is inhibited.

Obviously, numerous modifications and variations of the present invention are possible in light of the above teachings. It is therefore to be understood that within the scope of the appended claims, the invention may be practiced otherwise than as specifically described herein.

What is claimed is:

1. An analysis carrier for trapping a biologically active substance, comprising:
a carrier body having a carrier surface on which at least one polymer is immobilized,
wherein the at least one polymer includes a first repeating unit and a second repeating unit, the first repeating unit has a side chain including a functional group of a betaine structure, and the second repeating unit has a side chain whose terminal group is an active ester group.

2. The analysis carrier according to claim 1, wherein the functional group of a betaine structure is a phosphorylcholine group.

3. The analysis carrier according to claim 1, wherein the at least one polymer is in a form of a layer which is formed on the carrier surface by a process comprising introducing a polymerizable functional group or a chain transfer group to a surface of a carrier substrate material, mixing the carrier substrate material with a polymerizable monomer mixture including a first polymerizable monomer and a second polymerizable monomer, and performing a polymerization reaction such that the first polymerizable monomer forms the first repeating unit, and that the second polymerizable monomer forms the second repeating unit.

4. The analysis carrier according to claim 3, wherein the first polymerizable monomer comprises a monomer of Formula 1: 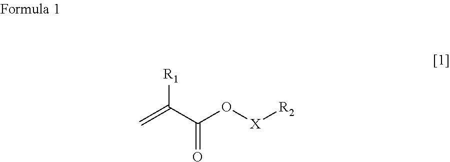
where R₁ represents a hydrogen atom or a methyl group, R₂ represents a functional group having a betaine structure, and X represents a hydrocarbon chain of between 0 and 20 carbons, which may be interrupted by —O—, —S—, —NH—, —CO—, or —CONH—.

5. The analysis carrier according to claim 3, wherein the first polymerizable monomer is 2-(meth)acryloyloxyethyl phosphorylcholine.

6. The analysis carrier according to claim 3, wherein the second polymerizable monomer comprises a monomer of Formula 2: 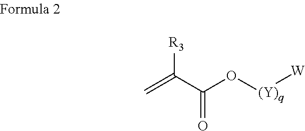
where R₃ represents a hydrogen atom or a methyl group, Y represents a 1 to 10 carbon alkylene glycol residue or an alkylene group, W represents an active ester group, q is an integer between 1 and 100, and when q is an integer between 2 and 100, repeating Y may be the same or different.

7. The analysis carrier according to claim 6, wherein the active ester group includes a p-nitrophenyl group or a succinimide group, and an ester bond.

8. The analysis carrier according to claim 3, wherein the polymerizable functional group is introduced to the carrier surface, and the polymerizable functional group is at least one of a methacryl group, an acryl group, and a vinyl group.

9. The analysis carrier according to claim 3, wherein the chain transfer group is introduced to the carrier surface, and the chain transfer group is a mercapto group.

10. The analysis carrier according to claim 1, wherein the carrier body comprises a carrier substrate material which is an inorganic material.

11. The analysis carrier according to claim 10, wherein the inorganic material is an inorganic oxide.

12. The analysis carrier according to claim 11, wherein the inorganic oxide is silicon oxide.

13. The analysis carrier according to claim 1, wherein the carrier body is in a form of a granule, a substrate, a fiber, a filter, a film, or a sheet.

14. The analysis carrier according to claim 3, wherein the introducing of a polymerizable functional group or a chain transfer group to the carrier surface comprises forming a covalent bond between a silane coupling agent having a polymerizable functional group or a chain transfer group and a functional group of the carrier surface serving as a nucleus.

15. The analysis carrier according to claim 14, wherein the silane coupling agent is an alkoxysilane having a polymerizable functional group or a chain transfer group.

16. A method of manufacturing an analysis carrier, comprising:
mixing in a solvent a carrier substrate material and a polymerizable monomer such that at least one polymer is formed from the polymerizable monomer and immobilized on a surface of the carrier substrate material; and
drying the carrier substrate material having the at least one polymer,
wherein the carrier substrate material has a polymerizable functional group or a chain transfer group introduced to the surface before the mixing with the polymerizable monomer, the at least one polymer includes a first repeating unit and a second repeating unit, the first repeating unit has a side chain including a functional group of a betaine structure, and the second repeating unit has a side chain whose terminal group is an active ester group.

17. The method according to claim 16, wherein the polymerizable functional group or the chain transfer group is introduced by a process comprising hydrolyzing an alkoxysilane having the polymerizable functional group or the chain transfer group in an acid aqueous solution, and stirring, under heating, the carrier substrate material in an acid aqueous solution including the alkoxysilane.

18. The method according to claim 16, wherein the at least one polymer is formed by a radical polymerization reaction.

19. A method of trapping a biologically active substance on an analysis carrier, comprising:
contacting the analysis carrier of claim 1 with at least one of a solution, a blood, a blood plasma, a blood serum, a disrupted cell suspension, a cell culture liquid, and a tissue fractionation liquid such that a target biological substance is collected by the analysis carrier.

20. The method according to claim 19, wherein the analysis carrier includes the at least one polymer in a form of a layer formed on the carrier surface, and the contacting comprises causing the target biological substance to be immobilized on the analysis carrier via the active ester group of the layer.

21. The method according to claim 19, wherein the biologically active substance comprises at least one substance which is at least one protein selected from the group consisting of an enzyme, an antibody, a lectin, a receptor, protein A, protein G, protein A/G, avidin, streptavidin, NeutrAvidin, glutathione-S-transferase, and a glycoprotein; a peptide; an amino acid; a hormone; a nucleic acid; at least one sugar chain selected from the group consisting of a sugar, oligosaccharide, polysaccharide, sialic acid derivative, and sialylated carbohydrate chain; a lipid; a low molecular weight compound; an organic polymer substance; or an inorganic substance,
a cointegrate of the at least one substance, or
a molecule forming a virus or a cell.

22. The method according to claim 19, wherein the analysis carrier includes the at least one polymer in a form of a layer formed on the carrier surface, and the contacting comprises contacting a solution in which the target biological substance is dissolved in a phosphate buffer solution with the analysis carrier.

23. The method according to claim 22, wherein the phosphate buffer solution has a phosphate concentration of between 0.1 M and 5 M.

24. The method according to claim 22, wherein the phosphate buffer solution includes at least one of potassium dihydrogenphosphate, sodium dihydrogenphosphate, dipotassium hydrogenphosphate, and disodium hydrogenphosphate.
